(19) United States
(12) Patent Application Publication
(10) Pub. No.: US 2004/0148013 A1
(43) Pub. Date: Jul. 29, 2004

(54) Stent-based delivery statins to prevent restenosis

(76) Inventors: Stephen E Epstein, Rockville, MD (US); Shmuel Fuchs, Rockville, MD (US); Eugenio Stabile, Washington, DC (US)

Correspondence Address: HELLER EHRMAN WHITE & MCAULIFFE LLP, 1666 K STREET,NW, SUITE 300, WASHINGTON, DC, 20006, US

(21) Appl. No.: 10/476,215

(22) PCT Filed: Apr. 18, 2002
(86) PCT No.: PCT/US02/12120

(51) Int. Cl. A61F 2/06

(52) U.S. Cl. 623/1.15; 623/1.42

(57) ABSTRACT

Restenosis of arteries after angioplasty in inhibited by implanting in the treated artery a stent (100) having the struts (102) coated with a composition including a statin (106) having anti-restenotic activity. Such statins may also be incorporated in a collagen or polymer matrix (106) that forms a coating covering the struts (102) and interstices (104) of the stent (100).

### RELATIONSHIP TO OTHER APPLICATIONS

This application claims the benefit of copending U.S. Provisional Patent Application No. 60/286,519, filed Apr. 27; 2001, the entire disclosure of which is incorporated herein by reference.

### BACKGROUND OF THE INVENTION

1. Field of the Invention

This invention relates to the prevention of restenosis of arteries after angioplasty and more particularly to the use of a stent platform which is coated with a composition including a statin compound, the effect of which is to prevent such restenosis.

2. Brief Description of the Prior Art

Coronary angioplasty has become an important method of treating narrowed (stenotic) arteries supplying the heart or the legs. Although the initial success rate of coronary angioplasty for opening obstructed coronary arteries exceeds 95%, restenosis occurs at the site of angioplasty in 25-50% of patients within six months, regardless of the type of angioplasty procedure used. Although the use of stents has appreciably reduced the rate of stenosis, even with this treatment strategy restenosis occurs in 5 to 20% of patients. Importantly, when restenosis occurs within a stent, the chance that restenosis will recur is very high. Thus, the problem of restenosis is still formidable, despite recent advances in reducing its incidence.

Two primary mechanisms appear to be involved in the development of restenosis. First, recoil of the vessel wall (negative remodeling) leads to gradual narrowing of the vessel lumen. Second, an exaggerated healing response of medial and/or adventitial smooth muscle cells (SMCs) to vascular injury involves the excessive proliferation of SMCs and the migration of SMCs to the subintima, where they continue to proliferate and begin to secrete extracellular matrix. These processes involving SMCs cause the neointimal mass to expand and gradually encroach upon the coronary lumen. Ultimately the expanding lesion narrows the vessel, increases the resistance to blood flow, and causes ischemic symptoms. In the absence of stenting, both remodeling and an expanding neointima contribute to restenosis. When stents are deployed, negative vascular remodeling is prevented and restenosis occurs only as a result of the expanding neointimal mass. Given these pathophysiologic mechanisms, the problem of controlling restenosis occurring with stent deployment becomes largely the problem of controlling the development of the neointimal mass.

Many attempts have been made to prevent the development of restenosis, and with the notable exception of brachytherapy, many such attempts have been reported to be successful in inhibiting neointima development in various experimental models. However, almost invariably their translation to clinical-interventions has been without success. These strategies have included the oral administration of drugs, their systemic administration, and their local delivery.

Local Delivery:

Therapeutic strategies began to focus on local delivery as it became apparent that high concentrations of active agent were needed at the target site. It would be very unlikely that such high concentrations could be achieved by any other approach than local delivery. Unfortunately, despite years of development and testing, the consensus is that catheter delivery systems are too inefficient to provide a high probability of success. Only one percent or less of the delivered product appears to persist for any period of time in the vessel wall.

The concept that drugs could be incorporated into stent coatings has become popularized, with mixed results. Most studies have shown no effect. However, preliminary encouraging results using stents having a coating impregnated with either Taxol® or its derivatives, or rapamycin, have been reported at several international meetings.

The success of these drugs is based on the cellular and molecular effects on microtubular modulation of the response of cells to mitogens and cytokines, and proteins controlling progress of cells through the cell-cycle.

Proteins controlling progress of cells through the cell-cycle: SMCs within the vessel wall are normally in a quiescent state. Immediately after injury, however, early response genes are expressed and the cells enter the cell cycle, wherein their replication is tightly regulated by an array of cell cycle regulatory proteins acting conjointly and in sequence at various points of the cycle. These regulatory proteins include cyclin-dependent kinases (cdc2 and cdk2), which phosphorylate critical regulatory proteins, and which interact with cyclin-dependent kinase inhibitors, such as p16, p21, and p27^{kip1}. Changes in the levels of these inhibitors exert marked effects on cell cycle progression, through inhibition of critical phosphorylation reactions.

One of the proteins involved in cell cycle progression that is regulated by phosphorylation is the tumor suppressor protein retinoblastoma protein (Rb). In the hypophosphorylated state Rb complexes with DNA binding and gene activating proteins, such as E2F, thereby exerting an inhibiting effect on cell cycle progression in G₀/mid G₁. Upon phosphorylation, the Rb/E2F complex dissociates, freeing E2F to bind to its DNA binding sites and consequently stimulate the transcription of genes inducing progression to the S phase of the cell cycle.

Rapamycin, a macrolide antibiotic, is a potent inhibitor of cell proliferation. It has recently been shown in a pig coronary artery injury model to significantly reduce the neointimal response to injury. The mechanism of action of rapamycin almost certainly largely derives from its ability to interfere with cell cycling. Thus, down-regulation of p27 by mitogens is blocked by rapamycin. Consistent with this activity, in the porcine injury model, rapamycin administration was associated with increased p27 levels and inhibition of Rb phosphorylation within the vessel wall. The most likely relevant molecular mechanisms are as follows: After binding to its cytosolic receptor, FKBP12, rapamycin increases p27, reduces cdc2 and cdk2 activity, and inhibits Rb phosphorylation, thereby inhibiting release of E2F. Eliminating E2F activity blocks the E2F-mediated transcription of the broad array of genes that contribute to cell cycle progression.

Microtubular modulation of the response of cells to mitogens and cytokines: The microtubular system has been shown to modulate the response of cells to various mitogens and cytokines through activation of transmembrane signaling cascades. Targets of these pathways include activation of kinases, including mitogen-activated protein kinase activity, changes associated with microbular depolymerization. The microtubules have also shown to play a part in the changes in SMCs that lead to their contributing to the restenosis lesion.

Paclitaxel favors stabilization of microtubule assembly, forming numerous disorganized microtubules within the cytoplasm, and thereby inhibiting many of the microtubular mediated cell signaling cascades cited above, including inhibition of cell division, predominantly in the G₀/G₁ and G₂/M phases of the cell cycle. Importantly, paclitaxel in biologically relevant concentrations does not appear to induce apoptosis. Taxol® inhibited, in vitro, both platelet-derived growth factor-stimulated SMC migration and SMC proliferation, and in vivo, inhibited neointimal accumulation in the rat carotid artery injury model.

The disappointing results of most strategies designed to inhibit restenosis, whether administered systemically or locally via catheter, and the initial promising results of agents administered with a stent-based delivery platform, emphasize the continued need to develop new agents to prevent restenosis, with an emphasis on delivering high local levels of the agent. It appears that the most promising strategy to achieve the latter goal is to deliver a potent anti-restenosis agent via a stent-based delivery system. A stent-based delivery system is disclosed in copending International Patent Application No. PCT/US01/45755, filed on Dec. 7, 2001, designating the United States, the entire disclosure of which is incorporated herein by reference. That application disclosed a method of preventing restenosis using a stent coated with DNA coding for gene products with anti-restenosis activity or cells containing such DNA. However, that application did not disclose using a stent coated with particular small molecules capable of exercising anti-restenosis activity on SMCs.

Accordingly, a need has continued to exist for additional methods of preventing restenosis using a stent coated with a substance having anti-restenosis activity on the cells of a blood vessel that has been treated by an angioplastic procedure.

### SUMMARY OF THE INVENTION

An advance in the treatment of restenosis after angioplasty has been achieved by this invention wherein a stent implanted in the treated artery is coated with a composition incorporating a statin compound (or DNA or other vector containing DNA encoding a molecule with statin-like activity). Alternatively, the statin may be incorporated into a matrix which is supported by the coating on the stent structure.

Accordingly, it is an object of the invention to provide a method for preventing or alleviating restenosis of an artery after angioplasty.

A further object is to provide a stent for implantation into an artery after angioplasty that is coated with a composition comprising a a source of a statin compound capable of delivering high local concentrations of the statin.

A further object is to provide a stent for implantation into an artery after angioplasty that is coated with a composition comprising a very high percentage by weight of a statin compound.

Further objects will be apparent from the description of the invention which follows.

### BRIEF DESCRIPTION OF THE DRAWINGS

FIG. 1 illustrates an uncoated or bare stent of the type implanted in an artery after angioplasty to inhibit restenosis.

FIG. 2 is a schematic illustration of the stent of FIG. 1 coated with a composition containing a statin.

FIG. 3 is a schematic illustration of an enlarged cross-section of a strut of the coated stent of FIG. 2, taken along the line 3-3 in FIG. 2.

FIG. 4 is a schematic illustration of the stent of FIG. 1 coated with a collagen gel that fills the areas between the struts and releasably contains a statin compound.

### DETAILED DESCRIPTION OF THE INVENTION AND PREFERRED EMBODIMENTS

The invention comprises 1) a delivery system comprising a stent and a stent coating that is impregnated with a selected statin (or DNA or other vector containing DNA encoding a molecule with statin-like activity), and, correspondingly, 2) contacting the arterial wall adjacent the stent with a high dosage of a statin (or DNA or other vector containing DNA encoding a molecule with statin-like activity), thus inhibiting restenosis of the stent-treated artery. The strategy described herein has the benefits of substantially reducing the incidence of restenosis with minimal incidence of untoward complications, a result that has not been achieved by other anti-restenosis strategies whose results have been limited or, as with radiation therapy, carry unknown future risks.

The statin compounds useful in the method of the invention are natural and/or synthetic compounds that are known to have the physiological effect of lowering serum cholesterol levels in human patients. The class of statin compounds is well-known to those skilled in the art. Such compounds are typically mevalonate derivatives that limit cholesterol biosynthesis by inhibiting the enzyme 3-hydroxy-3-methylglutaryl coenzyme A reductase (HMG-COA reductase). Statins useful in the method of the invention include, but are not limited to, lovastatin, pravastatin, simvastatin, atorvastatin, fluvastatin, cerivastatin, and the like. Other molecules having statin-like activity may also be used in the method and coated stent of the invention. It is also according to the invention to incorporate into the stent coating DNA or a DNA-containing vector capable of transfecting target cells (smooth muscle cells or other cells) and coding for the production of statins or statin-like compounds within those cells. Consequently, the invention includes a method of preventing restenosis using a stent coated with a source of a statin compund that provides a dose of a statin compund to the SMCs of the blood vessel wall, either directly, by releasing a statin to act on the SMCs, or indirectly, by causing cells of the vessel wall adjacent to the stent to produce a statin compound.

According to this invention, the delivery systems utilized in contacting the arterial wall with a composition including a statin, may take several forms.

In a first embodiment, the delivery system comprises a stent covered by a composition including the selected statin, which adheres to the surface of the stent, thereby facilitating the delivery of the statin within the injured vessel wall, or to cells that are migrating from the media and/or adventitia to form the neointima. The coating can be formed from any material that can cover the surface of the stent and that has the above characteristics. One such candidate coating has been created by the Photolink® process of the SurModics Company (Eden Prairie, Minn.).

Within the first embodiment or strategy of the invention, two alternatives may be used:

1. A statin is incorporated into the stent coating, which covers the stent struts but not intervening spaces.

2. The stent coating acts as a support scaffolding for the binding of collagen, or a similarly appropriate polymer, to the stent. The collagen or polymer will provide a matrix for the statin that will allow complete coverage of the vessel wall. This interation of the invention would be particularly appropriate for vein grafts, which have no side branches. Hence, the complete coverage for the vessel wall will not result in side branch occlusion. The completely covered stent will facilitate two important features of the invention.

a. It will provide efficient contact between the statin and all of that part of the vessel in which the stent is deployed so that a greater percentage of the, cells within the vessel wall will be in tight apposition to the statin.

b. It will provide a collagen or polymer barrier to cells migrating from the media or adventitia on their way to form the expanding neointima.

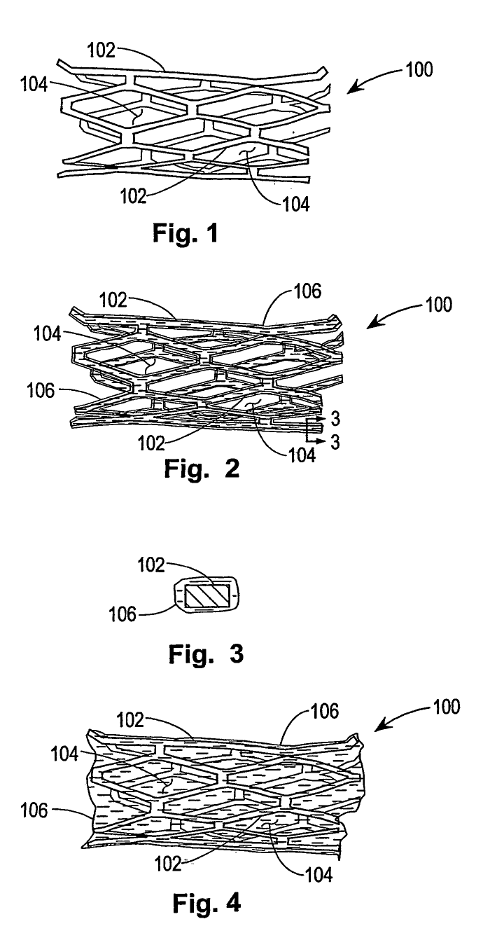

FIG. 1 illustrates the bare stent 100 without coating.

The stent comprises struts 102 having interstices or openings 104 between them.

FIG. 2 illustrates the stent 100 with a coating 106 releasably incorporating a statin compound. The coating 106 covers the metal struts 102 but not the intervening spaces 104.

FIG. 3 is a greatly enlarged view of a cross-section of a portion of the stent 100 of FIG. 2, taken along the line 3-3 in FIG. 2, showing the coated strut 102 and coating 106.

FIG. 4 illustrates the stent 100 of FIG. 1 provided with a coating of collagen 110 releasably containing a statin compound. The stent 100 serves as a scaffold for supporting the collagen gel 110 and statin compound incorporated into it.

The coating of the collagen gel 110 supported by the stent 100 covers not only the metal struts 102 (which cover only 15-20% of the arterial wall over which the stent extends), but also the intervening spaces or interstices 104, providing total coverage of the arterial wall.

Those skilled in the art will recognize that besides collagen other polymeric matrices capable of suspending the statins on the stent struts themselves, or of filling the interstices between the struts of the stent other coatings can be used, provided that they exhibit the necessary compatibility with the statin and permit release of the active agents to the adjacent artery wall or to cells migrating through the matrix. The properties of many such natural or synthetic polymeric matrices are well known or can be determined without undue experimentation to determine their suitability for use in the stent of this invention.

As previously stated, according to the invention, a statin (or DNA or other vector containing DNA encoding a molecule with statin-like activity) will be incorporated into a stent coating. The stent coating will consist of a substance that adheres to the stent, and which will incorporate the statin molecule without damaging it. It is expected that the platform system will facilitate delivery of the molecule to the cells within the injured vessel wall (or to the cells that are migrating from the media and/or adventitia to form the neointima), and is applied to the injured vessel wall at the time of angioplasty and stent implantation. This can be performed in any artery or interposed vein (such as, but not limited to, a saphenous vein graft to a coronary artery) that is obstructed and thereby impairs blood flow to the target tissue (whether it be heart or leg). The invention will employ any coating that can be attached to a stent and that has the above characteristics, and any molecule related chemically or functionally to a statin or DNA or other vector containing DNA encoding a molecule with statin-like activity. One such candidate coating has been created by the Photolink® process of SurModics. Oral formulations of many different statin molecules have been clinically tested; any of these, or those still being developed or that will be developed, are candidate molecules for this invention.

The intimate and prolonged contact between the injured vessel wall and a statin (or DNA or other vector containing DNA encoding a molecule with statin-like activity) that is contained within the stent coating and released therefrom, will lead to high local levels of the statin. This will exert the desired therapeutic effects on the cells contained within the vessel wall, such as, but not limited to, inhibition of smooth muscle cell (SMC) proliferation or migration and induction of SMC apoptosis.

These in vitro effects have in vivo parallels. Statins have antiproliferative effects on SMCs in acute vascular injury in nonatherosclerotic, normocholesterolemic rats and rabbits, and when administered orally to rats significantly reduce neointimal formation both after simple arterial injury and, importantly, after arterial stenting. This activity is completely reversed by simultaneous local administration of mevalonate which, as indicated above, supports a role of protein prenylation inhibition in these statin-induced actions.

The effect of statins on the development of restenosis and clinical outcome after coronary stent implantation has been assessed, but only in a retrospective study. Statin therapy was associated with a significant reduction in repeat target vessel revascularization procedures during 6-month follow-up. Minimal lumen diameter was significantly larger, late lumen loss was significantly less, and net gain significantly increased in patients receiving statin therapy. Dichotomous angiographic restenosis (>50%) rates were significantly lower, with 25% in the statin group compared with 38% in the no-statin group.

Importance of high dose on anti-restenotic effect: One of the findings of the study (referred to above) is of particular relevance to the current invention, which in a preferred embodiment comprises administering high concentrations of a selected statin to the potential restenotic site by stent-based delivery. Thus, it was found that whereas a low oral dose of the statin reduced cholesterol and decreased neointimal response to injury, a higher dose, despite no further effect on lipid lowering, resulted in a highly significant reduction in neointima formation.

Also, as indicated above, the replication of SMC replication is tightly regulated by cell cycle regulatory proteins including cyclin-dependent kinases (cdc2 and cdk2), which move cells through the cell cycle, and cyclin-dependent kinase inhibitors (such as p16, p21, and p27^{kip1}), which inhibit progression of cells through the cell cycle. Thus, Rapamycin, one of the few pharmacologic agents tested for which there are encouraging (albeit preliminary) clinical results suggesting a beneficial effect in limiting restenosis, is believed to act largely by its ability to block the down-regulation of p27 induced by various mitogens. This effect is accompanied by increased p27 levels, which inhibit Rb phosphorylation and thereby inhibit the release of Rb-bound E2F, a transcription factor responsible for stimulating the expression of a broad array of genes leading to cell cycle progression.

The small GTPases: The statins also exert molecular effects on cell-cycling proteins, but their effects are targeted to the small GTPases (ras, rho, etc), which are upstream effectors of the cell cycle regulatory proteins. Rho mediates cell cycle progression by down-regulating the expression of the cdk inhibitor p27^{kip1}, thereby leading to increased activity of cdk2 and hyperphosphorylation of Rb, which consequently causes release of E2F and E2F-mediated cell cycle progression. Post translational isoprenylation of these small GTPases is critical to their function by leading to their translocation to the cell membrane.

Cellular effects of statins that modulate proliferation and apoptosis independent of cholesterol-lowering effects: Statins competitively inhibit HMG-CoA reductase and thereby reduce cellular levels of mevalonate, precursor of the isoprenoids. Isoprenoids cause the prenylation of the small GTPases noted above. HMG-COA reductase inhibitors decrease rho geranylgeranylation and membrane translocalization, thereby preventing down-regulation of p27^{kip1} expression, which leads to increased activity of cdk2 and hyperphosphorylation of Rb. This consequently inhibits release of E2F and E2F-mediated cell cycle progression, thereby inhibiting SMC proliferation. These cellular effects also may be linked to the demonstrated induction of SMC programmed cell death (apoptosis) and inhibition of SMC migration caused by the statins. These pro-apoptotic and anti-proliferative and migratory effects are fully reversed by mevalonate, supporting a role of protein prenylation inhibition in these statin-induced actions. Such activities, if affecting SMCs located in the injured vessel wall, would reduce neointimal growth of developing restenotic lesions.

The invention, having now been fully described, should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than the foregoing description, and all changes which come within the meaning and range of equivalency of the claims are intended to be embraced therein.

We claim:

1. A method for inhibiting restenosis of blood vessels after angioplasty comprising
providing a stent comprising a lattice of interconnected struts with openings between said struts,
preparing a coated stent by coating at least a portion of said struts with a composition containing a source of a statin compound, capable of supplying said statin compound to cells adjacent to said coated stent, and
positioning said coated stent adjacent to a wall of a lumen of a blood vessel in conjunction with an angioplasty procedure.

2. The method of claim 1 wherein said source of a statin compound is selected from the group consisting of a statin compound, DNA coding for production of a statin compound, and a vector containing DNA coding for the production of a statin compound.

3. The method of claim 1 wherein said composition containing a source of a statin compound is a natural or synthetic polymer.

4. The method of claim 3 wherein said natural or synthetic polymer forms a layer covering at least a portion of said struts and said openings.

5. The method of claim 3 wherein said polymer is collagen.

6. The method of claim 4 wherein said polymer is collagen.

7. An intravascular stent comprising a lattice of interconnected struts with openings between said struts said struts being at least partially coated with a composition containing a source of a statin compound and capable of releasing said stating compound to adjacent smooth muscle cells.

8. The stent of claim 7 wherein said source of a statin compound is selected from the group consisting of a statin compound, DNA coding for production of a statin compound, and a vector containing DNA coding for the production of a statin compound.

9. The stent of claim 7 wherein said composition containing a source of a statin compound is a natural or synthetic polymer.

10. The stent of claim 9 wherein said natural or synthetic polymer forms a layer covering at least a portion of said struts and said openings.

11. The stent of claim 9 wherein said polymer is collagen.

12. The stent of claim 10 wherein said polymer is collagen.
